(19) United States
(12) Patent Application Publication
(10) Pub. No.: US 2022/0008307 A1
(43) Pub. Date: Jan. 13, 2022

(54) TWO-COMPONENT SYSTEM FOR ARTIFICIAL HAIR DYEING

(71) Applicant: Henkel AG & Co. KGaA, Duesseldorf (DE)

(72) Inventors: RENE KROHN, Norderstedt (DE); ERIK SCHULZE ZUR WIESCHE, Bielefeld (DE); ELISABETH POPPE, Hamburg (DE)

(73) Assignee: Henkel AG & Co. KGaA, Duesseldorf (DE)

(21) Appl. No.: 17/288,866

(22) PCT Filed: Oct. 31, 2019
(86) PCT No.: PCT/EP2019/079779

§ 371 Date: Apr. 26, 2021

(30) Foreign Application Priority Data
Oct. 31, 2018 (DE) 10 2018 127 182.4

(51) Int. Cl. A61K 8/58; A61K 8/31; A61K 8/34; A61K 8/41; A61K 8/19; A61Q 5/10

(52) U.S. Cl. CPC A61K 8/585; A61K 8/31; A61K 8/342; A61K 8/41; A61K 2800/4324; A61Q 5/10; A61K 2800/31; A61K 2800/52; A61K 8/19

(57) ABSTRACT

A two-component system for coloring keratinous material, wherein the two-component system separately comprises an anhydrous carrier medium comprising an alkane, a fatty alcohol and an alcohol, and an aqueous phase which in turn contains hydrogen peroxide. The carrier medium preferably contains certain organic silicon compounds.

### CROSS-REFERENCE TO RELATED APPLICATION

This application is a U.S. National-Stage entry under 35 U.S.C. § 371 based on International Application No. PCT/EP2019/079779, filed Oct. 31, 2019, which was published under PCT Article 21(2) and which claims priority to German Application No. 10 2018 127 182.4, filed Oct. 31, 2018, which are all hereby incorporated in their entirety by reference.

### TECHNICAL FIELD

The present disclosure relates to a two-component system for artificially coloring keratinous material, wherein the two-component system separately comprises an anhydrous carrier medium comprising an alkane, a fatty alcohol and an alcohol, and an aqueous phase which in turn contains hydrogen peroxide. Furthermore, the present disclosure relates to the use of the two-component system for artificial coloring or artificial coloring and care of hair.

### BACKGROUND

Changing the shape and color of keratinous material, especially human hair, is an important area of modern cosmetics. To change the hair color, the expert knows various coloring systems depending on the coloring requirements. Oxidation dyes are usually used for permanent, intensive dyeing with good fastness properties and good grey coverage. Such colorants contain oxidation dye precursors, so-called developer components and coupler components, which, under the influence of oxidizing agents such as hydrogen peroxide, form the actual dyes among themselves. Oxidation dyes are characterized by very long-lasting dyeing results.

When direct dyes are used, ready-made dyes diffuse from the colorant into the hair fiber. Compared to oxidative hair dyeing, the dyeing's obtained with direct dyes have a shorter shelf life and quicker wash ability. Dyeing with direct dyes usually remain on the hair for a period of between about 5 and about 20 washes.

For short-term color changes on the hair it is known to use color pigments. Color pigments are generally understood to be insoluble, coloring substances. These are present undissolved in the form of small particles in the coloring formulation and are merely deposited on the outside of the hair fibers and on the skin surface. Therefore, they can usually be removed again without residue by a few washes with detergents containing surfactants. Various products of this type are available on the market under the name hair mascara.

If the user wants particularly long-lasting dyeing, the use of oxidative dyes has so far been his only option. The hair damage still associated with the use of oxidative dyes also has a negative effect on the user's hair. A lingering challenge, therefore, is to find alternative, high-performance coloring processes, especially those that exert a conditioning action to cause less damage to the hair.

All coloring methods imply chemical stress on the hair. The external exposure of hair to chemicals from a variety of different sources poses challenges for the development of cosmetic care products. Furthermore, often changing consumer desires regarding a certain color of the hair are associated with recurring chemical exposure of the hair. For example, hair coloring stresses the hair, due to which a special, intensive care may be necessary.

It is a challenge for hair product developers on the one hand to satisfy customer wishes regarding color and texture and on the other hand to protect the structure, especially the surface structure, of the hair. Hair coloring has been difficult to manage with gentle means in the past.

For the protection and care of hair, organosilicon compounds from the group of silanes comprising at least one hydroxyl group and/or hydrolyzable group are described in the prior art. Due to the presence of the hydroxy groups and/or hydrolyzable groups, the silanes are reactive substances that hydrolyze or oligomerize or polymerize in the presence of water. The oligomerization or polymerization of the silanes initiated by the presence of the water, when applied to a keratinous material, ultimately leads to the formation of a film that can exert a protective effect.

The problem with using organosilicon compounds is their instability to water. Aqueous systems for hair treatment are thus disadvantageous if they contain organic silicone compounds as active ingredients.

### BRIEF SUMMARY

In various embodiments, a two-component system for artificial coloring of keratinous material, especially hair, separately comprises
an anhydrous carrier medium as a first component, comprising
   a) at least one branched or linear C8-C30 alkane,
   b) at least one branched or linear C10-C30 fatty alcohol,
   c) at least one branched or linear C2-C8 monohydric alcohol,
   d) at least one branched or linear C2-C10 amine as alkalizing agent, and
   e) one or more dyes to change the color of the keratinous material, and
an aqueous phase as a second component comprising water and hydrogen peroxide.

In certain embodiments, the anhydrous carrier medium of the two component system further comprises organic silicon compounds of formula (I), formula (I)), and/or formula (IV). In formula (I)

R₁R₂N-L-Si(OR₃)ₐ(R₄)_{b} (I),
R₁, R₂ both represent a hydrogen atom,
L represents a linear, C₁-C₆-alkylene group, preferably a propylene group (—CH₂—CH₂—CH₂—) or an ethylene group (—CH₂—CH₂—),
   R₃, R₄ independently represent a methyl group or an ethyl group,
a stands for the number 1 to 3 and
b stands for the number 3−a.

In formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

R₅, R_{5′}, R_{5″}, R₆, R_{6′} and R_{6″} independently represent a C₁-C₆ alkyl group,

A, A′, A″, A′″ and independently represent a linear or branched C₁-C₂₀ divalent alkylene group,

R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

-(A″″)-Si(R₆″)_{d″}(OR₅″)_{c″} (III),

wherein R5″ and R6″ are defined above and wherein
c stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
   with the proviso that at least one of the residues from e, f, g, and h is different from 0.

In various embodiments, the organic silicon compound is present in the anhydrous carrier medium in an amount of from about 0.01 to about 10% by weight based on the total weight of the anhydrous carrier medium. In a preferred embodiment, the organic silicon compound is (3-aminopropyl)triethoxysilane.

In embodiments, the anhydrous carrier medium further comprises an organic silicon compound of formula (IV),

R₉Si(OR₁₀)ₖ(R₁₁)ₘ (IV),

wherein
R9 stands for a C1-C12 alkyl group;
R10 stands for a hydrogen or a C1-C6 alkyl group;
R11 stands for a C1-C6 alkyl group;
k is a number from 1 to 3; and
m is 3−k.

In a further embodiment, a method for coloring hair involves combining the separately contained first and second components and applying the combined components onto hair that is to be dyed or colored.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The problem underlying the present disclosure is to provide a system which serves as a basis for the preparation of an agent with which gentle hair coloring is possible, the system exhibiting a long shelf life. In particular, the agent to be produced should preserve or restructure the surface structure of hair during its use.

This task is solved by a two-component system for coloring keratinous material, in particular hair, wherein the two-component system comprises separately:
an anhydrous carrier medium as a first component, comprising
   a) at least one branched or linear C8-C30 alkane,
   b) at least one branched or linear C10-C30 fatty alcohol,
   c) at least one branched or linear C2-C8 monohydric alcohol,
   d) at least one branched or linear C2-C10 amine as alkalizing agent, and
   e) one or more dyes to change the color of the keratinous material, and
an aqueous phase as a second component comprising water and hydrogen peroxide.

The advantage of two-component systems is the provision of the possibility to store substances that react with each other separately. Only shortly before application of the two-component system can the components be combined to provide a ready-to-use agent. Thus, in the context of the present invention, the feature “comprising separately” is to be understood as meaning that the two components, i.e., the anhydrous carrier medium and the aqueous phase, are present in two compartments or two different containers spatially separated from each other, with the purpose of preventing them from mixing unintentionally.

According to a preferred embodiment of the present invention, the carrier medium serves as a base for providing an organosilicon compound. In the carrier medium, the organic silicon compound remains storage stable. The carrier medium can be combined with one or more aqueous phases as a second component, which contains active ingredients serving for hair coloring and is based on water. The fact that the carrier medium is then combined with one or the other aqueous phases only shortly before use means that the active ingredient organosilicon compound remains stable until use.

In the context of the present invention, “anhydrous” should preferably be understood to mean that water is not added to the aqueous carrier medium or that the aqueous carrier medium is not water-based. More preferably, the water content of the anhydrous carrier medium is less than 5% by weight, even more preferably less than 2% by weight, most preferably less than 1% by weight, based on the total weight of the anhydrous carrier medium. In the presence of small amounts of moisture, a small portion of the organic silicon compound can hydrolyze, and the hydrolysate is present in equilibrium with free water. This amount of water is preferably present in the amounts mentioned above.

By a keratinous material is meant hair, the skin, the nails (such as fingernails and/or toenails). Wool, furs, and feathers also fall under the definition of keratinous material.

Preferably, a keratinous material is understood to mean human hair, human skin, and human nails, in particular fingernails and toenails. Very preferably, keratinous material is understood to mean human hair, in particular head and/or beard hair.

According to a preferred embodiment of the present invention, the anhydrous carrier medium of the two-component system contains at least one organic silicon compound, namely the one or more compounds to be stabilized. Preferred organic silicon compounds are selected from silanes having one, two or three silicon atoms, wherein the organic silicon compound comprises one or more hydroxyl groups and/or hydrolyzable groups per molecule. By using organic silicon compounds in the anhydrous carrier, the organic silicon compounds are protected from hydrolysis.

Organic silicon compounds, alternatively called organosilicon compounds, are compounds which either have a direct silicon-carbon bond (Si—C) or in which the carbon is bonded to the silicon atom via an oxygen, nitrogen, or sulfur atom. The organic silicon compounds are compounds containing one to three silicon atoms. Organic silicon compounds preferably contain one or two silicon atoms.

The effect of organic silicon compounds concerns the protection and care of hair, especially the hair surface, when using the two-component system for coloring hair. The hydrolyzed silicon compounds, when combined with the aqueous phase, form a protective film on the hair surface and thus have a “repair” effect.

According to IUPAC rules, the term silane stands for a group of chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

According to the preferred embodiment, the anhydrous carrier medium of the two-component system contains at least one organic silicon compound, preferably selected from silanes having one, two or three silicon atoms, wherein the organic silicon compound comprises one or more hydroxyl groups or hydrolyzable groups per molecule.

In a most preferred embodiment, the anhydrous carrier medium of the two-component system comprises at least one organic silicon compound selected from silanes having one, two or three silicon atoms, wherein the organic silicon compound further comprises one or more basic groups and one or more hydroxyl groups or hydrolyzable groups per molecule.

This basic group can be, for example, an amino group, an alkylamino group or a dialkylamino group, which is preferably connected to a silicon atom via a linker. The basic group is preferably an amino group, a C₁-C₆ alkylamino group or a di-(C₁-C₆)-alkylamino group.

The hydrolyzable group(s) is (are) preferably a C₁-C₆ alkoxy group, especially an ethoxy group or a methoxy group. It is preferred when the hydrolyzable group is directly bonded to the silicon atom. For example, if the hydrolyzable group is an ethoxy group, the organic silicon compound preferably contains a structural unit R′R″R′″Si—O—CH₂—CH₃. The residues R′, R″ and R′″ represent the three remaining free valences of the silicon atom.

Particularly good results were obtained when the anhydrous carrier medium of the two-component system contains at least one organic silicon compound of formula (I) and/or (II).

The compounds of formulae (I) and (II) are organic silicon compounds selected from silanes having one, two or three silicon atoms, the organic silicon compound comprising one or more hydroxyl groups and/or hydrolysable groups per molecule.

In another very particularly preferred embodiment, the anhydrous carrier medium of the two-component system comprises at least one organic silicon compound of the formula (I) and/or (II),

R₁R₂N-L-Si(OR₃)ₐ(R₄)_{b} (I),

where
R₁, R₂ both represent a hydrogen atom,
L represents a linear C₁-C₆-alkylene group, preferably a propylene group (—CH₂—CH₂—CH₂—) or an ethylene group (—CH₂—CH₂—),
R₃, R₄ independently represent a methyl group or an ethyl group,
a stands for the number 3 and
   b stands for the number 0.

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
R5, R5′, R5″ independently represent a hydrogen atom or a C₁-C₆ alkyl group,
R6, R6′ and R6″ independently represent a C₁-C₆ alkyl group,
A, A′, A″, A′″ and A″″ independently represent a linear or branched C₁-C₂₀ divalent alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

(A″″)-Si(R₆″)_{d}″(OR₅″)_{c}″ (III),
c, stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
provided that at least one of e, f, g, and h is different from 0.

The substituents R₁, R₂, R₃, R₄, R₅, R₅′, R₅″, R₆, R₆′, R₆″, R₇, R₈, L, A′, A″″ and A″″ in the compounds of formula (I) and (II) are explained below as examples:
Examples of a C₁-C₆ alkyl group are the groups methyl, ethyl, propyl, isopropyl, n-butyl, s-butyl, and t-butyl, n-pentyl and n-hexyl. Propyl, ethyl, and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear two-band C₁-C₂₀ alkylene group include the methylene group (—CH₂—), the ethylene group (—CH₂—CH₂—), the propylene group (—CH₂—CH₂—CH₂—) and the butylene group (—CH₂—CH₂—CH₂—). The propylene group (—CH₂—CH₂—CH₂—) is particularly preferred. From a chain length of 3 C atoms, divalent alkylene groups can also be branched. Examples of branched two-band C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. In particular, the radicals R₁ and R₂ both represent a hydrogen atom.

In the middle part of the organic silicon compound is the structural unit or the linker -L-, which stands for a linear or branched, divalent C₁-C₂₀ alkylene group.

Preferably, -L- represents a linear, divalent C₁-C₂₀ alkylene group. Further preferably, -L- represents a linear divalent C₁-C₆ alkylene group. Particularly preferred -L stands for a methylene group (—CH₂—), an ethylene group (—CH₂—CH₂—), propylene group (—CH₂—CH₂—CH₂—) or butylene (—CH₂—CH₂—CH₂—CH₂—). In an embodiment, L stands for a propylene group (—CH₂—CH₂—CH₂—).

The organic silicon compounds of formula (I)

R₁R₂N-L-Si(OR₃)ₐ(R₄)_{b} (I),

carry the silicon-containing grouping —Si(OR₃)ₐ(R₄)_{b} at one end.

In the terminal structural unit —Si(OR₃)ₐ(R₄)_{b}, R₃ is hydrogen or C₁-C₆ alkyl group, and R₄ is C₁-C₆ alkyl group. R₃ and R₄ independently of each other represent a methyl group or an ethyl group.

Here a stands for an integer from 1 to 3, and b stands for the integer 3−a. If a stands for the number 3, then b is equal to 0. If a stands for the number 2, then b is equal to 1. If a stands for the number 1, then b is equal to 2.

The best care of claimed hair could be obtained if the anhydrous carrier medium contains at least one organic silicon compound of formula (I) or formula (II) in which the radicals R₃, R₄ independently represent a methyl group or an ethyl group.

Particularly well-suited organic silicon compounds of formula (I) are

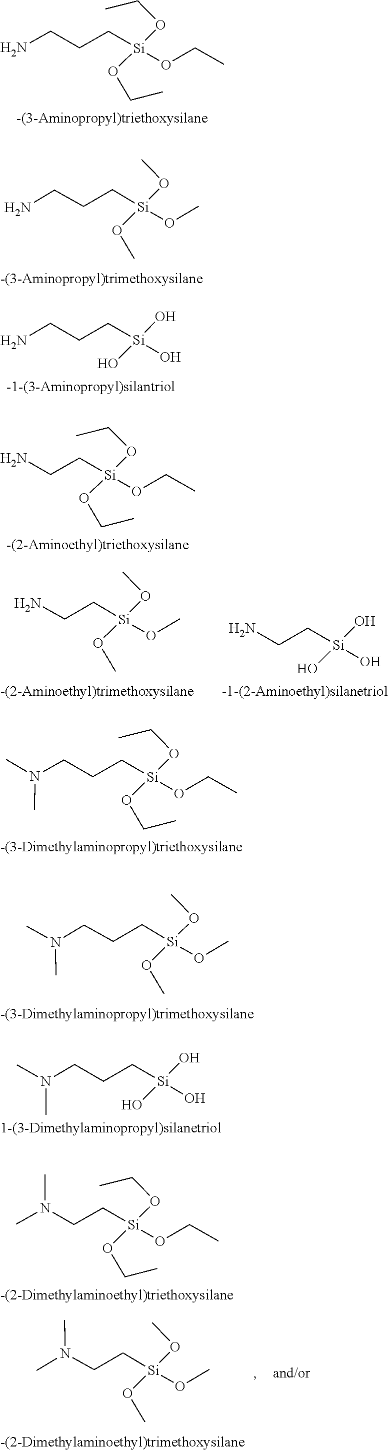

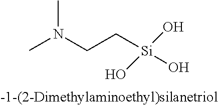

The organic silicon compound of formula (I) is commercially available. (3-aminopropyl)trimethoxysilane, for example, can be purchased from Sigma-Aldrich. (3-Aminopropyl)triethoxysilane is also commercially available from Sigma-Aldrich.

In another embodiment, the anhydrous carrier medium comprises at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II).

The organosilicon compounds of formula (II) each bear at their two ends the silicon-containing groupings (R₅O)_{c}(R₆)_{d}Si— and —Si(R₆′)_{d′}(OR₅′)_{c′}.

In the central part of the molecule of formula (II) there are the groups -(A)ₑ- and —[NR₇-(A′)]_{f}- and [O-(A″)]_{g}- and —[NR₈-(A′″)]ₕ-. Here, each of the radicals e, f, g, and h can independently of one another stand for the number 0 or 1, with the proviso that at least one of the radicals e, f, g, and h is different from 0. In other words, an organic silicon compound of formula (II) contains at least one grouping selected from the group consisting of -(A)- and —[NR₇-(A′)]- and —[O-(A″)]- and —[NR₈-(A′″)]-.

In the two terminal structural units (R₅O)_{c}(R₆)_{d}Sii- and —Si(R₆′)_{d′}(OR₅′)_{c}, the radicals R5, R5′, R5″ independently of one another represent a hydrogen atom or a C₁-C₆ alkyl group. The radicals R6, R6′ and R6″ independently represent a C₁-C₆ alkyl group.

Here a stands for an integer from 1 to 3, and d stands for the integer 3−c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3−c′. If c′ stands for the number 3, then d′ is 0. If c′ stands for the number 2, then d′ is 1. If c′ stands for the number 1, then d′ is 2.

Very preferred carrier media contain an organic silicon compound in which the radicals c and c′ both stand for the number 3. In this case d and d′ both stand for the number 0.

In another preferred one, the anhydrous carrier medium of the two-component system comprises at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
R5 and R5′ independently represent a methyl group or an ethyl group,
c and c′ both stand for the number 3 and
d and d′ both stand for the number 0.

When c and c′ both represent the number 3 and d and d′ both represent the number 0, the organic silicon compounds correspond to formula (IIa)

(R₅O)₃Si-(A)ₑ-[NR₇-(A′)]_{f}-[O-(A″)]_{g}-[NR8-(A′″)]ₕ-Si(OR₅′)₃ (IIa).

The radicals e, f, g, and h can independently stand for the number 0 or 1, whereby at least one radical from e, f, g, and h is different from zero. The abbreviations e, f, g, and h thus define which of the groupings -(A)ₑ- and —[NR7-(A′)]_{f}- and —[O-(A″)]_{g}- and —[NR8-(A′″)]ₕ- are in the middle part of the organic silicon compound of formula (II).

In this context, the presence of certain groupings has proven to be particularly beneficial in terms of increasing the care effect. Particularly good results were obtained when at least two of the residues e, f, g, and h stand for the number 1. Especially preferred e and f both stand for the number 1. Furthermore, g and h both stand for the number 0.

When e and f are both 1 and g and h are both 0, the organic silicon compounds are represented by the formula (IIb)

(R₅O)ₑ(R₆)_{d}Si-(A)-[NR₇-(A′)]-Si(R₆′)_{d′}(OR₅′)_{c′} (IIb).

The radicals A, A′, A″, A′″ and A″″ independently represent a linear or branched divalent C₁-C₂₀ alkylene group. Preferably the radicals A, A′, A″, A′″ and A″″ independently of one another represent a linear C₁-C₂₀ alkylene group. Further preferably the radicals A, A′, A″, A′″ and A″″ independently represent a linear divalent C₁-C₆ alkylene group. In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the residues A, A′, A″, A′″ and A″″ stand for a propylene group (—CH₂—CH₂—CH₂—).

When the radical f represents the number 1, the organic silicon compound of formula (II) contains a structural grouping —[NR₇-(A′)]-.

When the radical h represents the number 1, the organic silicon compound of formula (II) contains a structural grouping —[NR₈-(A′″)]-.

Wherein R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

-(A″″)-Si(R₆″)_{d}″(OR₅″)_{c}″ (III).

Very preferably, R₇ and R₈ independently represent a hydrogen atom, a methyl group, a 2-hydroxyethyl group, a 2-alkenyl group, a 2-aminoethyl group or a group of formula (III).

When the radical f represents the number 1 and the radical h represents the number 0, the organic silicon compound contains the grouping [NR₇-(A′)] but not the grouping —[NR₈-(A′″)]. If the radical R7 now stands for a grouping of the formula (III), the anhydrous carrier medium contains an organic silicon compound with 3 reactive silane groups.

In another preferred one, the anhydrous carrier medium of the two-component system comprises at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
e and f both stand for the number 1,
g and h both stand for the number 0,
A and A′ independently represent a linear, divalent C₁-C₆ alkylene group
   and
R7 represents a hydrogen atom, a methyl group, a 2-hydroxyethyl group, a 2-alkenyl group, a 2-aminoethyl group or a group of formula (III).

In another preferred embodiment, the anhydrous carrier medium of the two-component system comprises at least one organic silicon compound of the formula (II), wherein
e and f both stand for the number 1,
g and h both stand for the number 0,
A and A′ independently of one another represent a methylene group(—CH₂—), an ethylene group (—CH₂—CH₂—) or a propylene group (—CH₂—CH₂—CH₂),
   and
R₇ represents a hydrogen atom, a methyl group, a 2-hydroxyethyl group, a 2-alkenyl group, a 2-aminoethyl group or a group of formula (III).

Organic silicon compounds of formula (II) that are well suited for solving the problem are

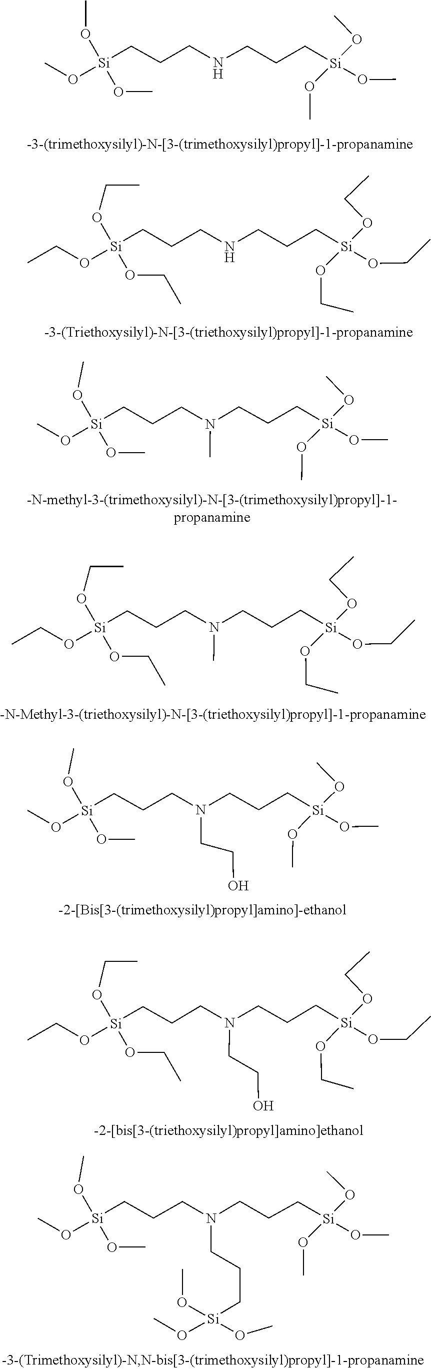

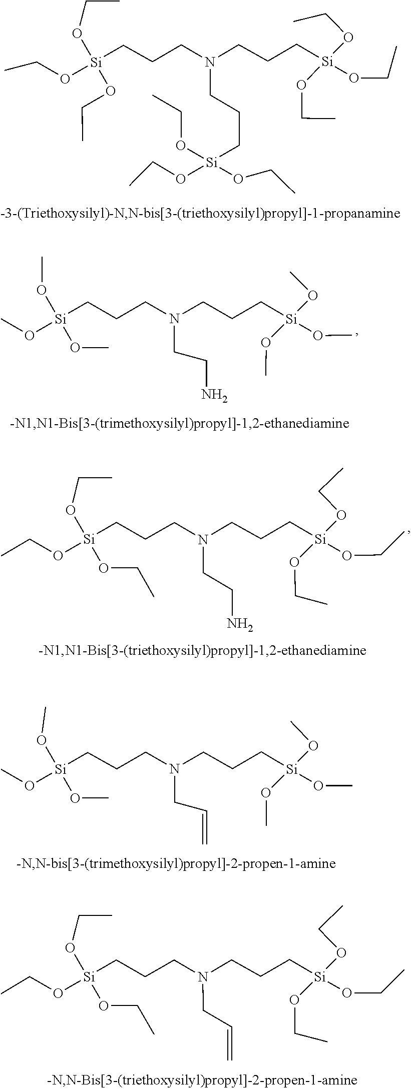

The organic silicon compounds of formula (II) are commercially available.

Bis(trimethoxysilylpropyl)amine with the CAS number 82985-35-1 can be purchased from Sigma-Aldrich.

Bis[3-(triethoxysilyl)propyl]amine, also known as 3-(triethoxysilyl)-N-[3-(triethoxysilyl)propyl]-1-propanamine, with CAS number 13497-18-2 kcan be purchased, for example, from Sigma-Aldrich or is commercially available under the product name Dynasylan 1122 from Evonik.

N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich or Fluorochem.

3-(triethoxysilyl)-N,N-bis[3-(triethoxysilyl)propyl]-1-propanamine with the CAS number 18784-74-2 can be purchased for example from Fluorochem or Sigma-Aldrich.

It has also been found to be advantageous if the two-component system applied to the hair contains at least one organic silicon compound of formula (IV)

R₉Si(OR₁₀)ₖ(R₁₁)ₘ (IV).

Thus, the anhydrous carrier medium also contains an organic silicon compound of formula (IV).

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be referred to as silanes of the alkylalkoxysilane or alkylhydroxysilane type,

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In a further preferred embodiment, the anhydrous carrier medium of the two-component system contains, in addition to the organic silicon compound or compounds of the formula (I), at least one further organic silicon compound of the formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In a likewise preferred embodiment, the anhydrous carrier medium of the two-component system contains, in addition to the organic silicon compound or compounds of the formula (II), at least one further organic silicon compound of the formula (IV) contains

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In a further preferred embodiment, the anhydrous carrier medium of the two-component system contains, in addition to the organic silicon compounds of the formula (I) and (II), at least one further organic silicon compound of the formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In the organic silicon compounds of formula (IV), the radical R₉ represents a C₁-C₁₂ alkyl group. This C₁-C₁₂ alkyl group is saturated and can be linear or branched. Preferably R9 stands for a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferably, R₉ represents a methyl group, an ethyl group or an n-octyl group.

In the organic silicon compounds of formula (IV), the radical R₁₀ represents a hydrogen atom or a C₁-C₆ alkyl group. Particularly preferably, R₁₀ represents a methyl group or an ethyl group.

In the organic silicon compounds of formula (IV), the radical R₁₁ represents a C₁-C₆ alkyl group. Particularly preferably, R₁₁ represents a methyl group or an ethyl group.

Furthermore, k stands for a whole number from 1 to 3, and m stands for the whole number 3−k. If k stands for the number 3, then m is equal to 0. If k stands for the number 2, then m is equal to 1. If k stands for the number 1, then m is equal to 2.

It has proved very advantageous for the anhydrous carrier medium to contain at least one organic silicon compound of the formula (IV) in which the radical k represents the number 3. In this case the rest m stands for the number 0.

Organic silicon compounds of the formula (IV) that are particularly suitable for solving the problem are

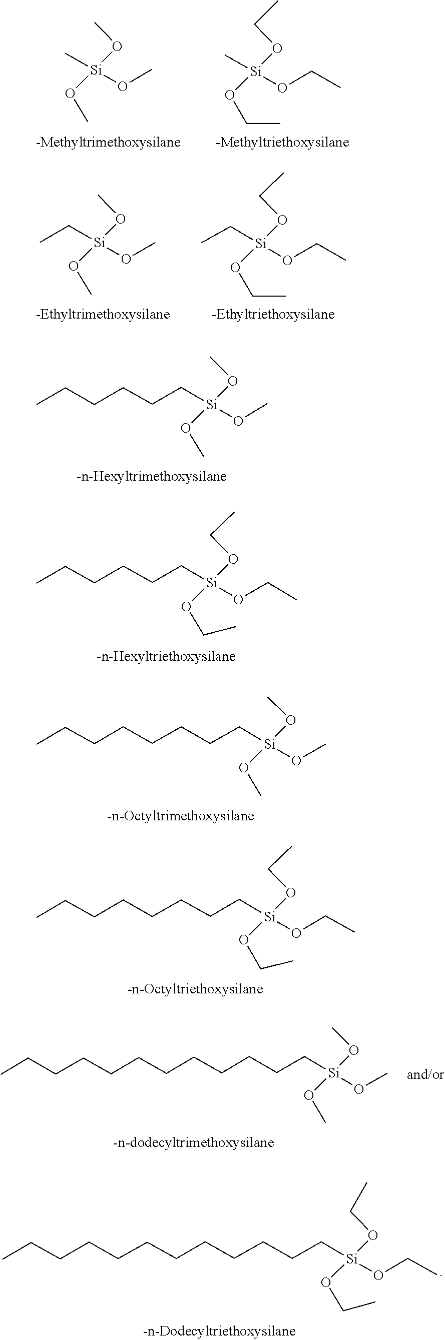

and propyltrimethoxysilane, propyltriethoxysilane, octadecyltrimethoxysilane and/or octadecyltriethoxysilane.

The organic silicon compounds described above are reactive compounds.

It was found that particularly stable and uniform films could be obtained on the keratinous material even when the anhydrous carrier medium contained two structurally different organic silicon compounds.

In a preferred embodiment, an anhydrous carrier medium is characterized in that it comprises at least one organic silicone compound of formula (I) and at least one organic silicone compound of formula (IV).

In an explicitly very particularly preferred embodiment, an anhydrous carrier medium is characterized in that it contains at least one organic silicone compound of formula (I) selected from the group consisting of (3-aminopropyl)triethoxysilane and (3-aminopropyl)trimethoxysilane, and additionally containing at least one organic silicone compound of formula (IV) selected from the group consisting of methyltrimethoxysilane, methyltriethoxysilane, ethyltrimethoxysilane, ethyltriethoxysilane, propyltrimethoxysilane, propyltriethoxysilane, hexyltrimethoxysilane and hexyltriethoxysilane.

In another preferred embodiment, an anhydrous carrier medium comprises—based on the total weight of the anhydrous carrier medium:
about 0.5 to about 5% by weight of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
about 3.2 to about 10% by weight of at least one second organic silicon compound selected from the group including methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane and octadecyltriethoxysilane.

Even the addition of small amounts of water leads to hydrolysis in organic silicon compounds with at least one hydrolyzable group. The hydrolysis products and/or organic silicon compounds having at least one hydroxy group may react with each other in a condensation reaction. For this reason, both the organosilicon compounds having at least one group that can be hydrolyzed and their hydrolysis and/or condensation products can be present in the anhydrous carrier medium. When organosilicon compounds having at least one hydroxyl group are used, both the organic silicon compounds having at least one hydroxyl group and their condensation products may be present in the anhydrous carrier medium.

A condensation product is understood to be a product formed by the reaction of at least two organic silicon compounds each having at least one hydroxyl group or hydrolyzable group per molecule with elimination of water and/or with elimination of an alkanol. The condensation products can be, for example, dimers, but also trimers or oligomers, with the condensation products being in equilibrium with the monomers. Depending on the amount of water used or consumed in the hydrolysis, the equilibrium shifts from monomeric organic silicon compounds to condensation product.

In the context of the present invention, figures in wt.-% are—unless otherwise stated—always based on the total weight of the anhydrous carrier medium. Where applicable, the figures are in -% by weight based on the total weight of the aqueous phase.

The second component of the two-component system of the present disclosure is an aqueous phase. Mandatorily, this second component contains water and hydrogen peroxide.

In the present invention, the hydrogen peroxide in the aqueous phase of the two-component system, after mixing the aqueous phase and anhydrous carrier medium, results in the two-component system being usable for hair shaping.

As components of the anhydrous carrier medium, five components are necessarily included in the anhydrous carrier medium: at least one branched or linear C8-C30 alkane, at least one branched or linear C10-C30 fatty alcohol, at least one branched or linear C2-C8 monohydric alcohol, at least one branched or linear C2-C10 amine as alkalizing agent, and one or more dyes to change the color of the keratinous material. In the course of the work leading to the present invention, it has been found that, to achieve a particularly good care effect, it is particularly advantageous if the five components together with the organic silicon compounds, for example 3-(triethoxysilyl)-N-[3-(triethoxysilyl)propyl]-1-propanamine, i.e., a bis(trimethoxysilylpropyl)amine, are combined in the anhydrous carrier medium with the aqueous phase. By excluding water in the anhydrous carrier medium, the organic silicon compound is protected from premature hydrolysis and is converted into a hair care emulsion only when required by mixing with a water phase, which activates the organic silicon compound by means of a hydrolysis reaction only shortly before application. Surprisingly, it was found that the combination of bis(trimethoxysilylpropyl)amine and the five mandatory components contained in the anhydrous carrier medium increased the cosmetic acceptability. The hair is soft, the combability is significantly increased and the hair surface is more hydrophobic in the chemically treated hair. At the same time a gentle coloring of the hair is achieved.

In the context of the present invention, the term “colorant for color change” is used for coloration of the keratin material, of the hair, caused using pigments and/or direct dyes. In this staining process, the colorant compounds are deposited in a particularly homogeneous and smooth film on the surface of the keratin material or diffuse into the keratin fiber. The term “dye for color change” can also be understood to mean a pigment. According to preferred embodiments, the two-component system also comprises a pigment.

Pigments within the meaning of the present disclosure are coloring compounds which have a solubility in water at 25° C. of less than 0.5 g/L, preferably less than 0.1 g/L, even more preferably less than 0.05 g/L. Water solubility can be determined, for example, by the method described below: 0.5 g of the pigment are weighed in a beaker. A stir-fish is added. Then one liter of distilled water is added. This mixture is heated to 25° C. for one hour while stirring on a magnetic stirrer. If undissolved components of the pigment are still visible in the mixture after this period, the solubility of the pigment is below 0.5 g/L. If the pigment-water mixture cannot be assessed visually due to the high intensity of the possibly finely dispersed pigment, the mixture is filtered. If a proportion of undissolved pigments remains on the filter paper, the solubility of the pigment is below 0.5 g/L. Suitable color pigments can be of inorganic and/or organic origin.

Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-containing silicates, silicates, metal sulfides, complex metal cyanides, metal sulphates, chromates and/or molybdates. Preferred color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, CI77510) and/or carmine (cochineal).

Colored pearlescent pigments are also particularly preferred color pigments. These are usually mica-based and may be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, mainly muscovite or phlogopite, is coated with a metal oxide.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

According to preferred embodiments of the present disclosure, the alkane in the two-component system is a C10-C24 alkane, preferably a C12-C18 alkane, and more preferably a C14-C16 alkane. Preferably, the alkane is present in the anhydrous carrier medium in an amount of 1 to 50% by weight, preferably from about 2 to about 45% by weight, more preferably from about 3 to about 40% by weight, even more preferably from about 4 to about 35% by weight, based on the total weight of the anhydrous carrier medium. A designation Cx to Cy always means that the hydrocarbon chain has x to y carbon atoms.

In a very particularly preferred embodiment of the present disclosure, the anhydrous carrier is an anhydrous concentrate. In the embodiment, the amount of alkane in the anhydrous carrier is from about 5 to about 90% by weight, preferably from about 10 to about 70% by weight, more preferably from about 15 to about 50% by weight.

According to another preferred embodiment of the present disclosure, the fatty alcohol is a C12-C24 fatty alcohol, preferably a C14-C18 fatty alcohol, wherein the fatty alcohol is preferably present in the anhydrous carrier medium in an amount of about 5 to about 50% by weight, more preferably 6 to 45% by weight, even more preferably from about 7 to about 40% by weight, most preferably from about 8 to about 35% by weight, based on the total weight of the anhydrous carrier medium.

According to another preferred embodiment of the present disclosure, the alcohol is a C3-C6 alcohol, preferably a C4-C5 alcohol, wherein the alcohol is preferably present in the anhydrous carrier medium in an amount of about 4 to about 50% by weight, preferably from about 6 to about 45% by weight, more preferably from about 8 to about 40% by weight, even more preferably from about 10 to about 30% by weight, based on the total weight of the anhydrous carrier medium. A monohydric alcohol is an alcohol with only one OH function.

According to another preferred embodiment of the present disclosure, the branched or linear amine is a C3-C8, more preferably a C4-C6 amine The amine is preferably present in the anhydrous carrier in an amount of from about 0.5 to about 10% by weight, more preferably from about 0.75 to about 7.5% by weight, even more preferably from about 1 to about 5% by weight, based on the total weight of the anhydrous carrier.

According to another preferred embodiment of the present disclosure, one or more dyes selected from the following group of dyes are included in the anhydrous carrier:
1,7-NAPHTHALENEDIOL, m-PHENYLENEDIAMINE, TOLUENE-2,5-DIAMINE SULFATE, 2,4-DIAMINOANISOL, p-PHENYLENEDIAMINE HCl, p-PHENYLENEDIAMINE (free base), 2-CHLORO-p-PHENYLENEDIAMINE, N-PHENYL-p-PHENYLENEDIAMINE, RESORCINOL, 4-CHLORORESORCINOL, o-AMINOPHENOL, m-AMINOPHENOL, p-AMINOPHENOL, 1-NAPHTHOL, 1,5-NAPHTHALENEDIOL, 2,7-NAPHTHALENEDIOL, HYDROQUINONE, p-METHYLAMINOPHENOL, p-METHYLAMINOPHENOLSULFATE, HYDROXYBENZOMORPHOLINE, 4-AMINO-2-HYDROXYTOLUENE, 2-METHYL-5-HYDROXYETHYLAMINOPHENOL, 1,2,4-TRIHYDROXYBENZENE, PHENYL METHYL PYRAZOLONE, 2,4-DIAMINOPHENOXYETHANOL HCl or SO4, 3-AMINO-2,4-DICHLOROPHENOL HCl, 2-METHYLRESORCINOL, N,N-BIS(2-HYDROXYETHYL)-p-PHENYLENEDIAMINE SULFATE, TETRAAMINOPYRIMIDINE SULFATE, 4-AMINO-m-CRESOL, 6-AMINO-m-CRESOL, 1,3-BIS-(2,4-DIAMINOPHENOXY)PROPANE×4 HCl, HYDROXYETHYL-p-PHENYLENEDIAMINE SULFATE, 2-AMINO-4-HYDROXYETHYLAMINOANISOLE SULFATE, 4,4-DIAMINODIPHENYLAMINE SULFATE, 5-AMINO-6-CHLORO-o-CRESOL, HYDROXYETHYL-3,4-METHYLENEDIOXYANILINE HCl, 2,6-DIHYDROXY-3,4-DIMETHYLPYRIDINE, 2,6-DIMETHOXY-3,5-PYRIDINEDIAMIE×(2) HCl, DIHYDROXYINDOLE, 2-AMINOMETHYL-p-AMINOPHENOL HCl, 2,4-DIAMINO-5-METHYLPHENETOL HCl, 5-AMINO-4-CHLORO-o-CRESOL HCl, HYDROXYPROPYL BIS(N-HYDROXYETHYL-p-PHENYLENEDIMAMINE) HCl, 6-HYDROXYINDOLE, ISATIN, 6-METOXY-2-METHYLAMINO-3-AMINOPYRIDINE HCl (HC BLUE 7), 2-AMINO-3-HYDROXYPYRIDINE, 2-DIMETHYLAMINO-5-AMINOPYRIDINE×2HCL, 6-METHOXY-2,3-PYRIDINEDIAMINE HCL, 2,6-DIAMINO PYRIDINE, 2,6-DIHYDROXYETHYLAMINOTOLUENE, 2,5,6-TRIAMINO-4_PYRIMIDINOL SULFATE, DIHYDROXYINDOLINE HBr, 1-ACETOXY-2-METHYLNAPHTHALENE, 1-HYDROXYETHYL 4,5-DIAMINOPYRAZOLE SULFATE, 2,2′-METHYLENEBIS 4-AMINOPHENOL HCl, 2-METHYL-1-NAPHTHOL, 4-Formyl 1-methylchinolinium-p-Toluene sulfonate, 2-AMINO-5-ETHYLPHENYL HCL, 2,3-DIAMINODIHYDROXY PYRAZOLOPYRAZOLONE DIMETHOSULFONATE, 2-METHOXY-METHYL-p-PHENYLENEDIAMINE, HYDROXYETHOXY AMINOPYRAZOLOPYRIDINE HCL, 3-AMINO-2,6-DIMETHYLPHENOL, 1-HEXYL 4,5-DIAMINO PYRAZOLE, SULFATE, DIMETHYLPIPERAZINIUM AMINOPYRAZOLO PYRIDINE, METHYLIMIDAZOLIUM p-PHENYLENEDIAMINE, ACID YELLOW 1, DISPERSE RED 17, BASIC BROWN 17, ACID BLACK 52, ACID BLACK 1, DISPERSE VIOLET 4, 4-NITRO-o-PHENYLENDIAMINE, 2-NITRO-P-PHENYLENEDIAMINE, 2-AMINO-4-NITROPHENOL, 2-AMINO-5-NITROPHENOL, PICRAMIC ACID and SODIUM PICRAMATE, HC RED NO. 13, N,N′-BIS(2-HYDROXYETHYL)-2-NITRO-p-PHENYLENEDIAMINE, HC YELLOW No 5, HC RED NO. 7, HC BLUE NO. 2, HC YELLOW NO. 4, HC YELLOW NO. 2, HC ORANGE NO. 1, HC RED NO. 1, HC RED NO. 3, 4-AMINO-3-NITROPHENOL, 2-HYDROXYETHYLAMINO-5-NITROANISOLE, 3-NITRO-p-HYDROXYETHYLAMINOPHENOL, 3 -METHYLAMINO-4-NITROPHENOXYETHANOL, 2-NITRO-5-GLYCERYL METHYLANILINE, HC VIOLET no 1, HC ORANGE no 2, HC YELLOW No 9, 4-NITROPHENYLAMINOETHYLUREA, HC RED NO. 10+HC RED NO. 11, 2-HYDROXYETHYL PICRAMIC ACID, HC BLUE NO. 12 as HCl, HC YELLOW NO. 6, HYDROXYETHYL-2-NITRO-p-TOLUIDINE, HC YELLOW NO. 12, HC BLUE No 11, HC YELLOW no 7, HC YELLOW no 10, 4-AMINO-2-NITRO-DIPHENYL-AMINE-2′-CARBOXYLIC ACID, 2-CHLORO-6-ETHYLAMINO-4-NITROPHENOL, HC VIOLET no 2, 2-AMINO-6-CHLORO-4-NITROPHENOL, 4-HYDROXYPROPYLAMINO-3-NITROPHENOL, HC YELLOW NO. 13, TETRAHYDRO-6-NITROQUINOXALINE, 2,6-DIAMINO-3-((PYRIDINE-3-YL)AZO)PYRIDINE, BASIC ORANGE 69, HC RED 16/N-(2-Nitro-4-aminophenyl)-allylamine, BASIC VIOLET 2 as HCl, BASIC RED 51, BASIC YELLOW 87, BASIC ORANGE 31, HC BLUE 16, HC RED No. 17, HC YELLOW 17, HC BLUE 18, HC YELLOW 16, HC RED 18, HC ORANGE 6, BASIC BLUE 124, BASIC RED 76, BASIC BROWN 16, BASIC YELLOW 57, ACID ORANGE 7, ACID RED 33, ACID YELLOW 23, ACID BLUE 9, ACID RED 92, “ACID YELLOW 3 (mono and di sodium)”, BASIC BLUE 99, ACID VIOLET 43, DISPERSE VIOLET 1, DISPERSE BLUE 3, ACID BLUE 62, DISPERSE BLACK 9, HYDROXYANTHRAQUINONE AMINOPROPYL METHYL MORPHOLINIUM METHOSULFATE, LAWSONE, LAWSONIA INERMIS, INDIGOFERA TINCTORIA, HC BLUE no 14, CURRY RED, ACID RED 18, ACID RED 52, ACID GREEN 25, DISPERSE BLUE 377, PIGMENT RED 57, HC BLUE No 15, TETRABROMOPHENOL BLUE, HC BLUE No 17 and/or BISMUTH CITRATE. For these dyes, the dyes with the INCI designation are meant. The amount of dye used depends very much on the type of dye. As a guide to the amount selected, it is indicated that about 0.1 to about 4% by weight of dye is present in the two-component system, based on the total weight of the two-component system.

According to preferred embodiments, the two-component system comprises emulsifiers to enable its components, i.e., the anhydrous carrier medium and the aqueous phase, to be homogenized and form a homogeneous phase during the preparation of the cosmetic composition ready for use. According to these preferred embodiments, the emulsifier or emulsifiers are present either in the aqueous phase or in the anhydrous carrier medium, but preferably in the aqueous phase.

A preferred emulsifier used is an alkyl trimonium compound having one or more C8-C22, more preferably C10-C18, even more preferably C12-C16 alkyl groups. An alkyltrimonium compound is a nitrogen-containing compound which is cationic, and which carries at least one, preferably several, organic radicals. The number of organic residues plus hydrogen always results in the number four.

Another preferred emulsifier is a fatty alcohol ethoxylate in which the fatty alcohol portion of the fatty alcohol ethoxylate has an alkyl chain length of C4-C30, preferably C6-C25, more preferably C8-C20, and/or in which the number of ethoxy groups in the fatty alcohol ethoxylate is from 2 to about 120, preferably from 4 to about 100, more preferably from 6 to about 80, even more preferably from about 8 to about 60, most preferably from about 10 to about 40.

Furthermore, a preferred emulsifier is a fatty alcohol sulfate with a chain length of C8-C22, preferably of C10-C20, more preferably of C12-C18.

According to further preferred embodiments, the emulsifiers are present in fixed amounts either in the anhydrous carrier medium or in the aqueous phase. A preferred two-component system contains the alkyltrimonium compound in an amount of from about 0.1 to about 5% by weight, preferably from about 0.5 to about 4% by weight, more preferably from about 1 to about 3% by weight, based on the total weight of the aqueous phase, in the aqueous phase, or the alkyltrimonium compound in an amount of from about 0.1 to about 5% by weight, preferably from about 0.5 to about 4% by weight, more preferably from about 1 to about 3% by weight, based on the total weight of the anhydrous carrier medium, in the anhydrous carrier medium.

According to a further preferred embodiment, the fatty alcohol ethoxylate is present in an amount of from about 0.1 to about 10% by weight, preferably from about 0.25 to about 7.5% by weight, more preferably from about 0.5 to about 5% by weight., based on the total weight of the aqueous phase, in the aqueous phase, or the fatty alcohol ethoxylate is present in an amount of from about 0.1 to about 10% by weight, preferably from about 0.25 to about 7.5% by weight, more preferably from about 0.5 to about 5% by weight, based on the total weight of the anhydrous carrier medium, in the anhydrous carrier medium.

Furthermore, according to preferred embodiments, the fatty alcohol sulfate is in an amount of about 0.1 to about 10% by weight, preferably from about 0.25 to about 7.5% by weight, more preferably from about 0.5 to about 5% by weight, based on the Total weight of the aqueous phase contained in the aqueous phase, or the fatty alcohol sulfate, is in an amount of about 0.1 to about 10% by weight, preferably from about 0.25 to about 7.5% by weight, more preferably from about 0.5 to about 5% By weight, based on the total weight of the anhydrous carrier medium, contained in the anhydrous carrier medium.

According to a preferred embodiment of the present disclosure, the hydrogen peroxide is present in the aqueous phase in an amount of from about 1 to about 12% by weight, preferably from about 2 to about 10% by weight, more preferably from about 3 to about 8% by weight, based on the total weight of the aqueous phase.

According to a preferred embodiment of the present disclosure, the weight ratio of the anhydrous carrier medium to the aqueous phase in the two-component system is from about 1 to about 10 to about 10 to about 1, preferably from about 5 to about 1 to about 1 to about 5, more preferably from about 2 to about 1 to about 1 to about 2.

In principle, all physiologically compatible counterions can be used as counterions of the present components, which are present as salts.

The aqueous phase or anhydrous carrier medium may comprise other additional ingredients in further preferred embodiments of the present disclosure. The following ingredients are optional and in addition to the components described above in the two-component system.

In another preferred two-component system, the anhydrous carrier medium or aqueous phase contains a cationic surfactant, wherein the cationic surfactant is one of the following formula,

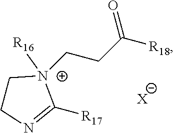

wherein
R₁₆ represents a C1-C6 alkyl group
R₁₇, R₁₈ independently represent a C7-C27 alkyl group, preferably a C10-C22 alkyl group, and
X− stands for a physiologically compatible anion,
   or wherein the cationic surfactant is one of the following formula,

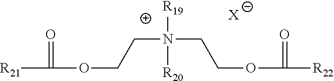

wherein
R₁₉, R₂₀ independently represent a C1-C6 alkyl group or a C2-C6 hydroxyalkyl group,
R₂₁, R₂₂ independently represent a C7-C27 alkyl group, preferably a C10-C22 alkyl group, and
X− stands for a physiologically compatible anion,
   or wherein the cationic surfactant is one of the following formula,

NR₂₃R₂₄R₂₅,

wherein
R₂₃, R₂₄ independently represent a C1-C6 alkyl group, a C2-C6 alkenyl group or a C2-C6 hydroxyalkyl group, and
R₂₅ represents a C8-C28 alkyl group, preferably a C10-C22 alkyl group,

The cationic surfactants of formula NR₂₃R₂₄R₂₅ are amine derivatives, so-called pseudoquats. The organic radicals R₂₃, R₂₄ and R₂₅ are directly bonded to the nitrogen atom. In the acidic pH range, these are cationized, i.e., the nitrogen atom is then protonated. The physiologically compatible counterions are suitable as counterions. Steamidopropyl dimethylamine is particularly preferred among these cationic surfactants.

In the present disclosure, the surfactants support the emulsification of aqueous phase and anhydrous carrier medium, or more generally the emulsification of water and oil phase. They are therefore optionally additionally included in the two-component system.

Further ingredients of the hair treatment compositions are described below which, in addition to the mandatory ingredients described above, may optionally be present in the anhydrous carrier medium or the aqueous phase.

According to further preferred embodiments, the anhydrous carrier medium or the aqueous phase further comprises a skin moisturizing or further skin care agent selected from the group consisting of glycerol, urea, hyaluronic acid, silanol esters of hyaluronic acid, panthenol, taurine, ceramides, phytosterols, aloe vera extracts, creatine, creatinine, sodium hyaluronate, polysaccharides, biosaccharides gum-1, cucumber extracts, butylene glycol, propylene glycol, methyl propanediol, ethylhexylglycerol, sorbitol, amino acids, glycine, glycine soy, histidine, tyrosine or tryptophan being particularly preferred amino acids, amino acid derivatives, natural betaine compounds, pyrrolidonecarboxylic acid or a salt of pyrrolidonecarboxylic acid, lactic acid, lactates, in particular sodium lactate, and/or ethylhexyloxyglycerol. In particular, the selection of these skin moisturizers increases the caring character of the anhydrous carrier medium.

According to another preferred embodiment, the anhydrous carrier medium or the aqueous phase contains several emulsifiers or several surfactants. It is particularly preferred that the anhydrous carrier medium or the aqueous phase contains two structurally different surfactants/emulsifiers, preferably the anhydrous carrier medium or the aqueous phase contains two structurally different cationic emulsifiers/surfactants, two anionic emulsifiers/surfactants which are different from one another, a cationic surfactant/emulsifier and a nonionic surfactant/emulsifier, or an anionic surfactant/emulsifier and a nonionic surfactant/emulsifier.

According to a preferred embodiment of the present disclosure, the cationic surfactant used comprises a hydrophobic head group with a cationic charge and one or two hydrophobic end portions, wherein the hydrophobic end portion(s) represent straight-chain or branched, saturated or mono- or polyunsaturated alkyl groups, preferably having a chain length of C6 to C30, more preferably C8 to C26, particularly preferably C10 to C22. According to another preferred embodiment, the cationic surfactant has an ester function, an ether function, a ketone function, an alcohol function, or an amide function.

According to a preferred embodiment of the present disclosure, the anhydrous carrier medium or aqueous phase contains as a further component a further nonionic surfactant, which preferably comprises a nonionic surfactant selected from the group including the following:
Alkylglucamide comprising a saturated or unsaturated, branched, or unbranched C₆ to C₂₂, preferably C₁₀ to C₁₈, more preferably C₁₂ to C₁₆ alkyl group,
Alkyl fructoside comprising a saturated or unsaturated, branched, or unbranched C₆ to C₂₂, preferably C₁₀ to C₁₈, more preferably C₁₂ to C₁₆ alkyl group,
An alkyl glucoside comprising a saturated or unsaturated, branched, or unbranched C₆ to C₂₂, preferably C₁₀ to C₁₈, more preferably C₁₂ to C₁₆ alkyl group, and
Alkyl alcohol alkoxylate of the formula R₁₀(OR₁₀)ₘOH, in which R₁₀ represents a linear or branched C₆-C₂₂, preferably C₁₀-C₁₈, more preferably C₁₂-C₁₆ alkyl group, R₁₁ represents a C₂-C₄, preferably a C₂ alkyl group, and m represents about 1 to about 10, preferably from about 2 to about 6, more preferably from about 2 to about 6,

According to preferred embodiments of the present disclosure, one or more further anionic surfactants are present as a component in the anhydrous carrier medium or aqueous phase, which is preferably selected from the group consisting of:
linear alpha-olefin sulfonates with 8 to 24, preferably 12 to 22, more preferably 16 to 18 C atoms,
straight-chain or branched, saturated or mono- or polyunsaturated alkylcarboxylic acids containing 8 to 24, preferably 12 to 22, more preferably 16 to 18 carbon atoms,
straight-chain or branched, saturated or mono- or polyunsaturated alkyl phosphates containing 8 to 24, preferably 12 to 22, more preferably 16 to 18 carbon atoms,
Alkyl isethionate whose alkyl group is selected from a branched or unbranched C₆ to C₂₂, preferably C₁₀ to C₁₈, more preferably C₁₂ to C₁₆ alkyl group, in particular sodium cocoyl isethionate,
Alkyl glycoside carboxylic acids whose alkyl group is selected from a branched or unbranched C₆ to C₂₂, preferably C₁₀ to C₁₈, more preferably C₁₂ to C₁₆ alkyl group,
Alkyl sulfosuccinates, the two alkyl groups of which are selected from identical or different, branched, or unbranched C₂ to C₁₂, preferably C₄ to C₁₀, more preferably C₆ to C₈ alkyl groups,
Alkyl taurates, the alkyl group of which is selected from a branched or unbranched C₆ to C₂₂, preferably C₁₀ to C₁₈, more preferably C₁₂ to C₁₆ alkyl group,
Alkyl sarcosinates whose alkyl group is selected from a branched or unbranched C₆ to C₂₂, preferably C₁₀ to C₁₈, more preferably C₁₂ to C₁₆ alkyl group,
Sulfonates of unsaturated fatty acids with 8 to 24, preferably 12 to 22, more preferably 16 to 18 C atoms and 1 to 6 double bonds,
   wherein the counterion of the anionic surfactant is an alkali or alkaline earth metal ion or a protonated triethanolamine or the ammonium ion.

Preferably, aqueous phase contains a surfactant mixture of anionic and amphoteric/zwitterionic surfactants sodium lauryl ether sulfate (INCI: sodium laureth sulfate) and very preferably sodium lauryl ether sulfate with 2 ethylene oxide units.

Amphoteric surfactants, also known as zwitterionic surfactants, are surface-active compounds that contain at least one quaternary ammonium group and at least one —COO⁻ or —SO₃⁻ group in the molecule Amphoteric/zwitterionic surfactants also include surface-active compounds which, in addition to a C₈-C₂₄ alkyl or acyl group, contain at least one free amino group and at least one —COOH— or —SO₃H— group and can form internal salts.

According to a preferred embodiment of the present disclosure, the anhydrous carrier medium or the aqueous phase contains at least one amphoteric surfactant as a further component. Preferably, the amphoteric surfactants in the anhydrous carrier medium are selected from the group consisting of
Alkyl betaine comprising at least one saturated or unsaturated, branched, or unbranched C₆ to C₂₂, preferably C₁₀ to C₁₈, more preferably C₁₂ to C₁₆ alkyl group,
Alkyl amphodiacetate or alkyl amphodiacetate comprising a saturated or unsaturated, branched, or unbranched C₆ to C₂₂, preferably C₁₀ to C₁₈, more preferably C₁₂ to C₁₆ alkyl group, with an alkali or alkaline earth metal counterion, and
Alkylamidopropyl betaine comprising at least one saturated or unsaturated, branched, or unbranched C₆ to C₂₂, preferably C₁₀ to C₁₈, more preferably C₁₂ to C₁₆ alkyl group.

Particularly suitable amphoteric/zwitterionic surfactants include those known under the INCI designation cocamidopropyl betaine and disodium cocoamphodiacetate.

In particular, the anhydrous carrier medium can be used to prepare an agent for coloring a keratinous material, an agent for conditioning a keratinous material, and/or an agent for conditioning and coloring a keratinous material.

It may be preferred that the anhydrous carrier medium or aqueous phase for treating a keratinous material further comprises about 0.001 to about 20% by weight of at least one quaternary compound. This applies to anhydrous carrier media or aqueous phases used for the preparation of an agent for the care of a keratinous material or for the care and cleaning of a keratinous material.

It is preferred that the at least one quaternary compound is selected from at least one of the groups consisting of:
i) the monoalkylquats and/or
ii) the esterquats and/or
iii) the quaternary imidazolines of formula (Tkat2),

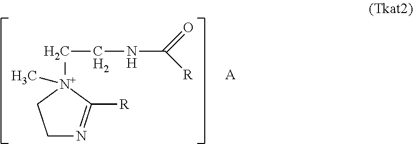

in which the radicals R independently of one another each represent a saturated or unsaturated, linear, or branched hydrocarbon radical having a chain length of 8 to 30 carbon atoms and A represents a physiologically tolerated anion, and/or
iv) the amidoamines and/or cationized amidoamines and/or
v) Poly(methacryloyloxyethyltrimethylammonium compounds) and/or
vi) quaternized cellulose derivatives, polyquaternium 10, polyquaternium-24, polyquaternium-27, polyquaternium-67, polyquaternium-72, and/or
vii) cationic alkyl polyglycosides and/or
viii) cationized honey and/or
ix) cationic guar derivatives and/or
x) Chitosan and/or
xi) polymeric dimethyldiallylammonium salts and copolymers thereof with esters and amides of acrylic acid and methacrylic acid, in particular polyquaternium-7 and/or
xii) Copolymers of vinylpyrrolidone with quaternized derivatives of dialkylaminoalkyl acrylate and methacrylate, especially polyquaternium-11 and/or
xiii) vinylpyrrolidone-vinylimidazolium methochloride copolymers, in particular polyquaternium-16 and/or
xiv) quaternized polyvinyl alcohol and/or
xv) Polyquaternium-74,
   and mixtures thereof.

It is preferred that the anhydrous carrier medium or aqueous phase contains a cationic homopolymer falling under the INCI designation polyquaternium-37 as quaternary compounds.

It may be preferred that the anhydrous carrier medium or aqueous phase further comprises a firming compound, preferably selected from the group consisting of waxes, synthetic polymers, and mixtures thereof.

To meet the different requirements of cosmetic agents used for the preparation of an agent for the treatment of a keratinous material for the temporary reshaping of a keratinous material (=styling agent), many synthetic polymers have already been developed as firming compounds which can be used in the agent for the treatment of a keratinous material. Alternatively, or complementarily, waxes are used as strengthening compounds. Ideally, the polymers and/or waxes, when applied to the keratinous material, result in a polymer film or sheet that, on the one hand, gives the hairstyle a strong hold, but, on the other hand, is sufficiently flexible not to break when stressed.

The synthetic polymers can be divided into cationic, anionic, nonionic, and amphoteric strengthening polymers.

Suitable synthetic polymers include, for example, polymers with the following INCI designations: Acrylamides/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/Ammonium Methacrylate Copolymer, Acrylates/t-Butyl Acrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/Hydroxyesters Acrylates Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopic Acid ntyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetone Acrylamide Copolymer, Ammonium-Acrylates/VA/Acrylates Copolymer, AMPD-Acrylates/Acrylamides Copolymer, AMPD-Acrylates/Acrylamides/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-hydroxypropyl Dimethicone Acrylate Copolymer, Isopropyl Ester of PVM/MA Acrylate Crosspolymer, Lauryl Methacrylate/Glycol Dimethacrylate Crosspolymer, MEA-Sulphite, Methacrylic Acid/Sodium Acrylamidomethyl Propane Sulphonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophen-2, Polyquaternium-2 Polyquaternium-4, Polyquaternium-5, Pol yquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, 18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polquaternium-56 9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 polyglyce ryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylbiloxylsilicon VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer. Cellulose ethers such as hydroxypropyl cellulose, hydroxyethyl cellulose and methyl hydroxypropyl cellulose are also suitable.

Also, homopolyacrylic acid (INCI: Carbomer), which is commercially available under the name Carbopol® in various forms, is suitable as a firming compound.

Preferably, the firming compound comprises a vinylpyrrolidone-containing polymer. Particularly preferably, the firming compound comprises a polymer selected from the group consisting of polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), and mixtures thereof.

Another preferred solidifying compound is octylacrylamide/acrylates/butylaminoethyl methacrylate copolymer (INCI), which is marketed under the name “Amphomer®” by Akzo Nobel.

Accordingly, it is particularly preferred that the firming compound comprises a synthetic polymer selected from the group consisting of polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), octylacrylamide/acrylates/butylaminoethyl methacrylate copolymer (INCI), and mixtures thereof.

The cosmetic compositions may contain, in addition or as an alternative to a synthetic polymer, at least one natural or synthetic wax having a melting point above 37° C. as a firming compound.

Natural or synthetic waxes can be solid kerosenes or isoparaffins, vegetable waxes such as candelilla wax, carnauba wax, esparto grass wax, Japan wax, cork wax, sugar cane wax, ouricury wax, montan wax, sunflower wax, fruit waxes and animal waxes such as beeswaxes and other insect waxes, Whale wax, shellac wax, wool wax and brushing grease, furthermore mineral waxes, such as ceresin and ozokerite or petrochemical waxes, such as petrolatum, kerosene waxes, microwaxes of polyethylene or polypropylene and polyethylene glycol waxes can be used. It may be advantageous to use hydrogenated or cured waxes. Chemically modified waxes, in particular hard waxes such as montan ester waxes, sasol waxes and hydrogenated jojoba waxes, can also be used.

Also suitable are the triglycerides of saturated and optionally hydroxylated C16-30 fatty acids, such as hydrogenated triglyceride fats (hydrogenated palm oil, hydrogenated coconut oil, hydrogenated castor oil), glyceryl tribehenate or glyceryl tri-12-hydroxystearate.

The wax components can also be selected from the group of esters of saturated, unbranched alkanecarboxylic acids having a chain length of 22 to 44 carbon atoms and saturated, unbranched alcohols having a chain length of 22 to 44 carbon atoms, provided that the wax component or the totality of wax components are solid at room temperature. Silicone waxes, for example stearyltrimethylsilane/stearyl alcohol, may also be beneficial.

Natural, chemically modified, and synthetic waxes can be used alone or in combination. However, this is not intended to include alkanes, which are necessarily present in the anhydrous carrier medium. Several waxes can also be used. Furthermore, several wax mixtures, possibly mixed with other additives, are also commercially available. The products sold under the designations “Special Wax 7686 OE” (a mixture of cetyl palmitate, beeswax, microcrystalline wax and polyethylene with a melting range of about 73-75° C.; manufacturer: Kahl & Co), Polywax® GP 200 (a mixture of stearyl alcohol and polyethylene glycol stearate with a melting point of about 47-51° C.; manufacturer: Croda) and “Softceresin® FL 400” (a vaseline/vaseline oil/wax mixture with a melting point of about 50-54° C.; manufacturer: Parafluid Mineral Oil Company) are examples of mixtures that can be used.

Preferably, the wax is selected from carnauba wax (INCI: Copernicia Cerifera Cera) Bienenwachs (INCI: Beeswax), petrolatum (INCI), microcrystalline wax and especially mixtures thereof.

Preferred blends include the combination of carnauba wax (INCI: Copernicia Cerifera Cera), petrolatum and microcrystalline wax or the combination of beeswax (INCI: Beeswax) and petrolatum.

The wax or wax components should be solid at 25° C. and should melt in the range >37° C.

The anhydrous carrier medium or the aqueous phase preferably contains the firming compound in a total amount of about 0.5 to about 50% by weight, preferably from about 1 to about 40% by weight, more preferably from about 1.5 to about 30% by weight, even more preferably from about 2 to about 25% by weight, based on the total weight of the cosmetic composition.

Other suitable ingredients include nonionic polymers, anionic polymers, (further) cationic polymers, waxes, protein hydrolysates, amino acids, oligopetides, vitamins, provitamins, vitamin precursors, betaines, biochinones, purine (derivatives), plant extracts, silicones, ester oils, UV light filters, structuring agents, thickening agents, electrolytes, pH-adjusting agents, swelling agents, colorants, anti-dandruff agents, complexing agents, opacifiers, pearlescent agents, pigments, stabilizing agents, propellants, antioxidants, perfume oils and/or preservatives.

Another object of the present application is the use of the two-component system for coloring a keratinous material or for maintaining and coloring a keratinous material.

About further preferred embodiments of use, what has been said about the anhydrous carrier media applies mutatis mutandis.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A two-component system for artificial coloring of keratinous material, separately comprising
an anhydrous carrier medium as a first component, comprising
a) at least one branched or linear C8-C30 alkane,
b) at least one branched or linear C10-C30 fatty alcohol,
c) at least one branched or linear C2-C8 monohydric alcohol,
d) at least one branched or linear C2-C10 amine as alkalizing agent, and
e) one or more dyes to change the color of the keratinous material, and
an aqueous phase as a second component comprising water and hydrogen peroxide.

2. The two-component system according to claim 1, wherein the anhydrous carrier medium further comprises at least one organic silicon compound which is a compound of formula (I) or of formula (II),
wherein in the organic silicon compound of formula (I)
R₁R₂N-L-Si(OR₃)ₐ(R₄)_{b} (I),
R₁, R₂ both represent a hydrogen atom,
L represents a linear, C₁-C₆-alkylene group,
R₃, R₄ independently represent a methyl group or an ethyl group,
a stands for the number 1 to 3 and
b stands for the number 3−a, and
wherein in the organic silicon compound of formula (II)
(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),
R₅, R_{5′}, R_{5″}, R₆, R_{6′} and R_{6″} independently represent a C₁-C₆ alkyl group,
A, A′, A″, A′″ and A″″ independently represent a linear or branched C₁-C₂₀ divalent alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)
-(A″″)-Si(R₆″)_{d″}(OR₅″)_{c″} (III),
c stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
with the proviso that at least one of the residues from e, f, g, and h is different from 0.

3. The two-component system according to claim 2,
wherein
that the anhydrous carrier medium comprises at least one organic silicon compound of formula (I) selected from the group consisting of
(3-Aminopropyl)trimethoxysilane
(3-Aminopropyl)triethoxysilane
(2-Aminoethyl)trimethoxysilane
(2-Aminoethyl)triethoxysilane
(3-Dimethylaminopropyl)trimethoxysilane
(3-Dimethylaminopropyl)triethoxysilane
(2-dimethylaminoethyl)trimethoxysilane and
(2-Dimethylaminoethyl)triethoxysilane.

4. The two-component system according to claim 2, wherein,
the two-component system comprises at least one organic silicon compound of the formula (II) which is selected from the group consisting of
3-(trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-Methyl-3-(triethoxysilyl)-N-[3-(triethoxysilyl)propyl]-1-propanamine
2-[Bis[3-(trimethoxysilyl)propyl]amino]-ethanol
2-[Bis[3-(triethoxysilyl)propyl]amino]ethanol
3-(Trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-Bis[3-(trimethoxysilyl)propyl]-1,2-ethanediamine,
N1,N1-Bis[3-(triethoxysilyl)propyl]-1,2-ethanediamine,
N,N-Bis[3-(trimethoxysilyl)propyl]-2-propen-1-amine and
N,N-Bis[3-(triethoxysilyl)propyl]-2-propen-1-amine.

5. Two-component system according to claim 1, wherein the organic silicon compound is present in the anhydrous carrier medium in an amount of from about 0.01 to about 10% by weight based on the total weight of the anhydrous carrier medium the organic silicon compound being (3-aminopropyl)triethoxysilane.

6. A two-component system according to claim 2, wherein the anhydrous carrier medium further comprises at least one organic silicon compound of formula (IV),
R₉Si(OR₁₀)ₖ(R₁₁)ₘ (IV),
wherein
R9 stands for a C1-C12 alkyl group;
R10 stands for a hydrogen or a C1-C6 alkyl group;
R11 stands for a C1-C6 alkyl goup;
k is a number from 1 to 3; and
m is 3−k.

7. The two-component system according to claim 1, wherein the alkane is a C10-C24 alkane, and wherein the alkane is present in the anhydrous carrier medium in an amount of from about 1 to about 50% by weight based on the total weight of the anhydrous carrier medium.

8. Two-component system according to claim 1, wherein the aqueous phase or the anhydrous carrier medium comprises one or more emulsifiers.

9. (canceled)

10. The two-component system according to claim 2, wherein the hydrogen peroxide is present in the aqueous phase in an amount of from about 1 to about 12% by weight, and wherein
the dye for color change is selected from the group consisting of
1,7-NAPHTHALENEDIOL, m-PHENYLENEDIAMINE, TOLUENE-2,5-DIAMINE SULFATE, 2,4-DIAMINOANISOL, p-PHENYLENEDIAMINE HCl, p-PHENYLENEDIAMINE (free base), 2-CHLORO-p-PHENYLENEDIAMINE, N-PHENYL-p-PHENYLENEDIAMINE, RESORCINOL, 4-CHLORORESORCINOL, o-AMINOPHENOL, m-AMINOPHENOL, p-AMINOPHENOL, 1-NAPHTHOL, 1,5-NAPHTHALENEDIOL, 2,7-NAPHTHALENEDIOL, HYDROQUINONE, p-METHYLAMINOPHENOL, p-METHYLAMINOPHENOLSULFATE, HYDROXYBENZOMORPHOLINE, 4-AMINO-2-HYDROXYTOLUENE, 2-METHYL-5-HYDROXYETHYLAMINOPHENOL, 1,2,4-TRIHYDROXYBENZENE, PHENYL METHYL PYRAZOLONE, 2,4-DIAMINOPHENOXYETHANOL HCl or SO4, 3-AMINO-2,4-DICHLOROPHENOL HCl, 2-METHYLRESORCINOL, N,N-BIS(2-HYDROXYETHYL)-p-PHENYLENEDIAMINE SULFATE, TETRAAMINOPYRIMIDINE SULFATE, 4-AMINO-m-CRESOL, 6-AMINO-m-CRESOL, 1,3-BIS-(2,4-DIAMINOPHENOXY)PROPANE×4 HCl, HYDROXYETHYL-p-PHENYLENEDIAMINE SULFATE, 2-AMINO-4-HYDROXYETHYLAMINOANISOLE SULFATE, 4,4-DIAMINODIPHENYLAMINE SULFATE, 5-AMINO-6-CHLORO-o-CRESOL, HYDROXYETHYL-3,4-METHYLENEDIOXYANILINE HCl, 2,6-DIHYDROXY-3,4-DIMETHYLPYRIDINE, 2,6-DIMETHOXY-3,5-PYRIDINEDIAMIE×(2) HCl, DIHYDROXYINDOLE, 2-AMINOMETHYL-p-AMINOPHENOL HCl, 2,4-DIAMINO-5-METHYLPHENETOL HCl, 5-AMINO-4-CHLORO-o-CRESOL HCl, HYDROXYPROPYL BIS(N-HYDROXYETHYL-p-PHENYLENEDIMAMINE) HCl, 6-HYDROXYINDOLE, ISATIN, 6-METOXY-2-METHYLAMINO-3-AMINOPYRIDINE HCl (HC BLUE 7), 2-AMINO-3-HYDROXYPYRIDINE, 2-DIMETHYLAMINO-5-AMINOPYRIDINE×2HCL, 6-METHOXY-2,3-PYRIDINEDIAMINE HCL, 2,6-DIAMINO PYRIDINE, 2,6-DIHYDROXYETHYLAMINOTOLUENE, 2,5,6-TRIAMINO-4_PYRIMIDINOL SULFATE, DIHYDROXYINDOLINE HBr, 1-ACETOXY-2-METHYLNAPHTHALENE, 1-HYDROXYETHYL 4,5-DIAMINOPYRAZOLE SULFATE, 2,2′-METHYLENEBIS 4-AMINOPHENOL HCl, 2-METHYL-1-NAPHTHOL, 4-Formyl 1-methylchinolinium-p-Toluene sulfonate, 2-AMINO-5-ETHYLPHENYL HCL, 2,3-DIAMINODIHYDROXY PYRAZOLOPYRAZOLONE DIMETHOSULFONATE, 2-METHOXY-METHYL-p-PHENYLENEDIAMINE, HYDROXYETHOXY AMINOPYRAZOLOPYRIDINE HCL, 3-AMINO-2,6-DIMETHYLPHENOL, 1-HEXYL 4,5-DIAMINO PYRAZOLE, SULFATE, DIMETHYLPIPERAZINIUM AMINOPYRAZOLO PYRIDINE, METHYLIMIDAZOLIUM p-PHENYLENEDIAMINE, ACID YELLOW 1, DISPERSE RED 17, BASIC BROWN 17, ACID BLACK 52, ACID BLACK 1, DISPERSE VIOLET 4, 4-NITRO-o-PHENYLENDIAMINE, 2-NITRO-P-PHENYLENEDIAMINE, 2-AMINO-4-NITROPHENOL, 2-AMINO-5-NITROPHENOL, PICRAMIC ACID and SODIUM PICRAMATE, HC RED NO. 13, N,N′-BIS(2-HYDROXYETHYL)-2-NITRO-p-PHENYLENEDIAMINE, HC YELLOW No 5, HC RED NO. 7, HC BLUE NO. 2, HC YELLOW NO. 4, HC YELLOW NO. 2, HC ORANGE NO. 1, HC RED NO. 1, HC RED NO. 3, 4-AMINO-3-NITROPHENOL, 2-HYDROXYETHYLAMINO-5-NITROANISOLE, 3-NITRO-p-HYDROXYETHYLAMINOPHENOL, 3-METHYLAMINO-4-NITROPHENOXYETHANOL, 2-NITRO-5-GLYCERYL METHYLANILINE, HC VIOLET no 1, HC ORANGE no 2, HC YELLOW No 9, 4-NITROPHENYLAMINOETHYLUREA, HC RED NO. 10+HC RED NO. 11, 2-HYDROXYETHYL PICRAMIC ACID, HC BLUE NO. 12 as HCl, HC YELLOW NO. 6, HYDROXYETHYL-2-NITRO-p-TOLUIDINE, HC YELLOW NO. 12, HC BLUE No 11, HC YELLOW no 7, HC YELLOW no 10, 4-AMINO-2-NITRO-DIPHENYL-AMINE-T-CARBOXYLIC ACID, 2-CHLORO-6-ETHYLAMINO-4-NITROPHENOL, HC VIOLET no 2, 2-AMINO-6-CHLORO-4-NITROPHENOL, 4-HYDROXYPROPYLAMINO-3-NITROPHENOL, HC YELLOW NO. 13, TETRAHYDRO-6-NITROQUINOXALINE, 2,6-DIAMINO-3-((PYRIDINE-3-YL)AZO)PYRIDINE, BASIC ORANGE 69, HC RED 16/N-(2-Nitro-4-aminophenyl)-allylamine, BASIC VIOLET 2 as HCl, BASIC RED 51, BASIC YELLOW 87, BASIC ORANGE 31, HC BLUE 16, HC RED No. 17, HC YELLOW 17, HC BLUE 18, HC YELLOW 16, HC RED 18, HC ORANGE 6, BASIC BLUE 124, BASIC RED 76, BASIC BROWN 16, BASIC YELLOW 57, ACID ORANGE 7, ACID RED 33, ACID YELLOW 23, ACID BLUE 9, ACID RED 92, “ACID YELLOW 3 (mono and di sodium)”, BASIC BLUE 99, ACID VIOLET 43, DISPERSE VIOLET 1, DISPERSE BLUE 3, ACID BLUE 62, DISPERSE BLACK 9, HYDROXYANTHRAQUINONE AMINOPROPYL METHYL MORPHOLINIUM METHOSULFATE, LAWSONE, LAWSONIA INERMIS, INDIGOFERA TINCTORIA, HC BLUE no 14, CURRY RED, ACID RED 18, ACID RED 52, ACID GREEN 25, DISPERSE BLUE 377, PIGMENT RED 57, HC BLUE No 15, TETRABROMOPHENOL BLUE, HC BLUE No 17 or BISMUTH CITRATE.

11. Two-component system according to claim 1, wherein the weight ratio of the anhydrous carrier medium to the aqueous phase is in the range from about 1 to 10 to about 10 to 1.

12. (canceled)

13. The two component system according to claim 6, wherein the anhydrous carrier medium comprises a silicon compound selected from
methyltrimethoxysilane;
methyltriethoxysilane;
ethyltrimethoxysilane;
ethyltriethoxysilane;
propyltrimethoxysilane;
propyltriethoxysilane;
hexyltrimethoxysilane;
hexyltriethoxysilane;
octyltrimethoxysilane;
octyltriethoxysilane;
dodecyltrimethoxysilane;
dodecyltriethoxysilane;
octadecyltrimethoxysilane; and
octadecyltriethoxysilane.

14. The two component system according to claim 2, wherein the anhydrous carrier medium comprises a silicon compound of Formula (I) and a silicon compound of Formula (II).

15. The two component system according to claim 14, wherein the anhydrous carrier medium further comprises a silicon compound of Formula (IV).

16. The two component system according to claim 6, wherein the anhydrous carrier medium comprises an organic silicone compound of formula (I) selected from the group consisting of (3-aminopropyl)triethoxysilane and (3-aminopropyl)trimethoxysilane, and additionally comprises an organic silicone compound of formula (IV) selected from the group consisting of methyltrimethoxysilane, methyltriethoxysilane, ethyltrimethoxysilane, ethyltriethoxysilane, propyltrimethoxysilane, propyltriethoxysilane, hexyltrimethoxysilane and hexyltriethoxysilane.

17. The two component system according to claim 2, wherein the anhydrous carrier medium comprises
0.5 to 5% by weight of at least one first organic silicon compound selected from the group consisting of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
3.2 to 10% by weight of at least one second organic silicon compound selected from the group consisting of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane and octadecyltriethoxysilane.

18. The two component system according to claim 1, wherein the anhydrous carrier medium comporises a C8-C30 alkane, a C10-C30 fatty alcohol, a C2-C8 monohydric alcohol, a C2-C10 amine, a dye, and bis(trimethoxysilylpropyl)amine.

19. A method for coloring hair, comprising combining a first component and a second component to provide a ready to use agent, and applying the ready to use agent onto keratin fibers to color those fibers, wherein
the first component comprises an anhydrous carrier medium comprising
at least one branched or linear C8-C30 alkane;
at least one branched or linear C10-C30 fatty alcohol;
at least one branched or linear C2-C8 monohydric alcohol;
at least one branched or linear C2-C10 amine as alkalizing agent; and
one or more dyes to change the color of the keratinous material, and
the second component is an aqueous phase comprising water and hydrogen peroxide.

20. The method according to claim 19, wherein the first component further comprises
(3-Aminopropyl)trimethoxysilane;
(3-Aminopropyl)triethoxysilane;
(2-Aminoethyl)trimethoxysilane;
(2-Aminoethyl)triethoxysilane;
(3-Dimethylaminopropyl)trimethoxysilane;
(3-Dimethylaminopropyl)triethoxysilane;
(2-dimethylaminoethyl)trimethoxysilane; or
(2-Dimethylaminoethyl)triethoxysilane.

21. The method according to claim 19, wherein the wherein the anhydrous carrier medium comprises
0.5 to 5% by weight of a first organic silicon compound selected from the group consisting of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
3.2 to 10% by weight of a second organic silicon compound selected from the group consisting of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane and octadecyltriethoxysilane.

22. The method according to claim 19, wherein the anhydrous carrier medium comprises bis(trimethoxysilylpropyl)amine.
